(19) United States
(12) Patent Application Publication
(10) Pub. No.: US 2021/0366606 A1
(43) Pub. Date: Nov. 25, 2021

(54) SYSTEM, METHOD AND COMPUTER PROGRAM PRODUCT FOR REMOTE MEASUREMENT OF VITAL SIGNS

(71) Applicant: Elta Systems Ltd., Ashdod (IL)

(72) Inventors: Avi Abraham SHAHADI, Kiryat Ono (IL); Ohad MIZRAHI, Ramat Gan (IL); Revital HUBER-SHALEM, Petach Tikva (IL); Sagi ZIMILIS, Petah Tikva (IL)

(21) Appl. No.: 17/235,335

(22) Filed: Apr. 20, 2021

(30) Foreign Application Priority Data
Apr. 20, 2020 (IL) 274078

(51) Int. Cl. G16H 40/67; G16H 50/20; A61B 5/0205; A61B 5/024; G01S 13/28; G01S 13/48; G01S 13/89

(52) U.S. Cl. CPC G16H 40/67; G16H 50/20; A61B 5/02055; G01S 13/89; G01S 13/282; G01S 13/48; A61B 5/02416

(57) ABSTRACT

A system for measuring medical data characterizing a subject to be monitored, the system including radar sensor/s and/or electro-optical sensor/s. The medical data, which may include pulse and/or respiratory rate and/or temperature of the subject to be monitored, are measured remotely, thereby providing standoff detection and reducing risk of infection to medical personnel.

### REFERENCE TO CO-PENDING APPLICATIONS

Priority is claimed from Israeli Patent Application No. 274078, entitled “System for remote measurement of vital signs” and filed Apr. 20, 2020, the disclosure of which application/s is hereby incorporated by reference.

### FIELD OF THIS DISCLOSURE

The present invention relates generally to measurements, and more particularly to measurements by radar systems inter alia.

### BACKGROUND FOR THIS DISCLOSURE

Radar systems aka radar sensors which detect heart-beat and include “single pixel”, short range, single beam radars, are known, such as:
www.researchgate.net/publication/241635913_Doppler_radar_for_heartbeat_rate_and_heart_rate_variability_extraction
www.nature.com/articles/s41598-018-29984-5
www.hindawi.com/journals/jhe/2018/4832605/

Radar systems aka radar sensors which measure respiration are known, e.g.
www.researchgate.net/publication/242602111_Wireless_bio-radar_sensor_for_heartbeat_and_respiration_detection
royalsocietypublishing.org/doi/full/10.1098/rsos.190149

patents.justia.com/patent/20180003554 describes use of two IR wavelengths for low false-alarm missiles flare detection; two cameras (or diode sensors) were used, one at IR, and one for visible light, at two different orientations). When a detection was made, one sensor was used for filtering sunlight, so as to reduce false alarms.

Vayyar produces mobile, low cost 3D imaging sensors.

The following publication: https://www.janes.com/article/95302/covid-19-israeli-defence-companies-adapt-sensors-to-screen-for-virus is a Janes.com publication dated 2 Apr. 2020, which describes “radar- and electro-optical-based sensors to monitor peoples' vital signs and identify es infected with Covid-19. . . . The data, including puke, respiratory rate and temperature, are measured remotely using a combination of radar and electro-optical sensors, thus reducing the risk of infection to medical personnel. The next stage of development [is] screening and prioritizing patient care based on the analysis of vital data” and describes “the need to monitor patients remotely before they come into contact with medical personnel in order to prevent infections.” The publication describes use of the IAI-Elta 1 kg ELM-2114 perimeter surveillance radar as the basis for a medical system and describes that “The radar uses the very low frequency K-band so can be safely placed very close to people. . . . Using the radar, the system can track a person's movements as they breathe, as well as their pulse when placed 3 m away, with medics in another room receiving readings in seconds.” Developers said they placed on a radar “the ability to measure pulse and breath rates . . . linked . . . with an electro-optic system that can measure body temperature” which “provides an alert, not a definite diagnosis, but . . . an indication.”

Saelig Company produces a Fotric 226B Infrared Thermal Imager, an example of a thermal camera which detects facial temperatures and is said to provide safe, non-contact measurement of passing human traffic.

ELTA's ELM-2112 and ELM-2114 are examples of staring radar families, used for ground and coastline surveillance, and HLS purposes.

Standoff detection of threats and dangers is a known general technological goal. For example, this publication:
www.photonics.com/Articles/Advances_in_Standoff_Detection_Make_the_World/a5 3439 describes Standoff detection of explosives, biological agents and hazardous chemical materials—without putting personnel and equipment in harm's way; such detection may take place at distances from several centimeters up to a kilometer from the threat, and may employ lasers and optical techniques.

The disclosures of all publications and patent documents mentioned in the specification, and of the publications and patent documents cited therein directly or indirectly, are hereby incorporated by reference other than subject matter disclaimers or disavowals. If the incorporated material is inconsistent with the express disclosure herein, the interpretation is that the express disclosure herein describes certain embodiments, whereas the incorporated material describes other embodiments. Definition/s within the incorporated material may be regarded as one possible definition for the term/s in question.

### SUMMARY OF CERTAIN EMBODIMENTS

Certain embodiments of the present invention seek to provide circuitry typically comprising at least one hardware processor in communication with at least one memory, with instructions stored in such memory executed by the processor to provide functionalities which are described herein in detail. Any functionality described herein may be firmware-implemented or processor-implemented, as appropriate.

Certain embodiments seek to provide a system for measuring medical data characterizing subject to be monitored, the system including at least one radar sensor and at least one electro-optical sensor, the at least one radar sensor and the at least one electro-optical sensor configured to remotely measure, and wherein the medical data, including pulse and/or respiratory rate and, optionally temperature of the subject, are measured remotely, thereby providing standoff detection and reducing the risk of infection to medical personnel.

Certain embodiments seek to provide radar- and electro-optical-based sensors to monitor peoples' vital signs e.g. to identify persons infected with Covid-19.

Typically, data, including all or any subset of pulse, respiratory rate and temperature, are measured remotely, using a combination of radar and electro-optical sensors, thus reducing the risk of infection to medical personnel. Analysis of data may include screening and prioritizing patient care. An advantage is the ability to monitor patients remotely before they come into contact with medical personnel in order to prevent infections of the medical staff. The system may include IAI-Elta's 1 kg ELM-2114 perimeter surveillance radar which is an example of a small radar system aka radar sensor. The radar typically uses an ISM (Industrial, Scientific, Medical) frequency band at K-band, at an EIRP (Effective Incident Radiated Power), typically according to suitable regulatory standards that can be safely placed very close to people. Using the radar, the system may track a person's movements as they breathe, as well as their pulse when placed, say, 3 m away, such that medics in another room may be receiving readings in seconds. The system adds to the radar, the ability to corroborate measurements of pulse and breath rates by an electro-optic system that may measure body temperature as well. The system may be used to provide a screening alert, rather than, necessarily, a definite diagnosis.

Thus at least the following embodiments a1, a2, . . . are provided:

Embodiment a1. A system or method or computer program product for measuring vital/crucial signs/parameters of a subject to be monitored, the system including (all or any subset of):

radar aka rf sensor,

thermal camera/s,

an optical sensor e.g. wide angle visible light camera; and

a hardware processor configured to process outputs of the above sensor/s.

Embodiment a2. The system of embodiment 1 wherein the parameters include at least one of heart rate, respiratory rate, and body temperature.

Embodiment a3. The system of any preceding embodiments and wherein heart rate aka heartbeat is measured by the rf sensor.

Embodiment a4. The system of any preceding embodiments and wherein respiration is measured by the RF sensor.

Embodiment a5. The system of any preceding embodiments and wherein respiration is measured by the thermal camera.

Embodiment a6. The system of any preceding embodiments and wherein respiration is measured by plural sensors (e.g. RF sensor and thermal camera/s).

Embodiment a7. The system of any preceding embodiments and wherein the hardware processor computes a respiratory rate by combining outputs of the RF sensor and thermal camera/s.

Embodiment a8. The system of any preceding embodiments and wherein body temperature is measured by the thermal camera/s.

Embodiment a9. The system of any preceding embodiments and wherein the body temperature includes readings for plural body parts, such as all or any subset of: forehead, hand, mouth, nostrils, neck.

Embodiment a10. The system of any preceding embodiments and wherein the optical sensor determines whether or not other sensors obtained their measurements while the monitored subject/objects were moving, thereby to facilitate processing of these measurements with higher success rates.

Embodiment a11. The system of any preceding embodiments and wherein at least one black body apparatus is used to provide calibration of the thermal camera's temperature outputs.

Embodiment a12. The system of any preceding embodiments and wherein the thermal camera/s detects temperature differences between inhaled and exhaled air temperature and time, thereby to quantify lung functioning.

Embodiment a13. The system of any preceding embodiments and also comprising a database which records outputs of the hardware processor in association with at least one of: monitored subject/subjects ID, testing facility ID, operator ID, and date/time of test.

Embodiment a14. The system of any preceding embodiments and also comprising software operative to monitor multiple stations.

Embodiment a15. The system of any preceding embodiments and wherein the hardware processor estimates lung tidal volume, as a function of at least one of:

cross sections of at least one of: nostrils, mouth, and

air flow rate determined as a function of temperature change rate measured by the thermal camera/s.

Embodiment a16. The system of any preceding embodiments and wherein the thermal camera's field of view includes at least one body portion whose temperature changes as a function of blood flow, and wherein heat changes resulting from the blood flow are sensed by the thermal camera, and wherein the hardware processor is configured to estimate heartbeat rate by computing the rate of the heat change as sensed, thereby to discern the heart rate at which blood through at least one artery adjacent the at least one body portion pulsate.

Embodiment a17. The system of any preceding embodiments and wherein the hardware processor uses A1 to influence the system itself in real time, including providing a learning system that updates itself repeatedly by measurement and corroboration by an external source of knowledge e.g. human medical team.

Embodiment a18. The system of any preceding embodiments and wherein the radar operates in staring radar mode.

Embodiment a19. The system of any preceding embodiments and wherein the thermal camera/s comprises plural thermal cameras having different wave lengths.

Embodiment a20. The system of any preceding embodiments and wherein heartbeat is measured by the thermal camera/s.

Embodiment a21. The system of any preceding embodiments and wherein heartbeat is measured by plural sensors (e.g. an RF sensor and thermal camera/s).

Embodiment a22. The system of any preceding embodiments and wherein the hardware processor computes a heartbeat rate by combining outputs of the RF sensor and thermal camera/s.

Embodiment a23. The system of any preceding embodiments wherein the lung functioning is quantified by computing lung tidal volume, in a noncooperative measurement.

Embodiment a24. The system of any preceding embodiments wherein the lung functioning is quantified by computing vital capacity and/or lung capacity, in a cooperative measurement.

It is appreciated that any reference herein to, or recitation of, an operation being performed is, e.g. if the operation is performed at least partly in software, intended to include both an embodiment where the operation is performed in its entirety by a server A, and also to include any type of “outsourcing,” or “cloud” embodiments, in which the operation, or portions thereof is or are performed by a remote processor P (or several such), which may be deployed off-shore or “on a cloud”, and an output of the operation is then communicated to, e.g. over a suitable computer network, and used by, server A. Analogously, the remote processor P may not, itself, perform all of the operations, and, instead, the remote processor P itself may receive output/s of portion/s of the operation from yet another processor/s P′, may be deployed off-shore relative to P, or “on a cloud”, and so forth.

Also provided, excluding signals, is a computer program comprising computer program code means for performing any of the methods shown and described herein when the program is run on at least one computer; and a computer program product, comprising a typically non-transitory computer-usable or -readable medium e.g. non-transitory computer-usable or -readable storage medium, typically tangible, having a computer readable program code embodied therein, the computer readable program code adapted to be executed to implement any or all of the methods shown and described herein. The operations in accordance with the teachings herein may be performed by at least one computer specially constructed for the desired purposes, or a general purpose computer specially configured for the desired purpose by at least one computer program stored in a typically non-transitory computer readable storage medium. The term “non-transitory” is used herein to exclude transitory, propagating signals or waves, but to otherwise include any volatile or non-volatile computer memory technology suitable to the application.

Any suitable processor/s, display and input means may be used to process, display e.g. on a computer screen or other computer output device, store, and accept information such as information used by or generated by any of the methods and apparatus shown and described herein; the above processor's, display and input means including computer programs, in accordance with all or any subset of the embodiments of the present invention. Any or all functionalities of the invention shown and described herein, such as but not limited to operations within flowcharts, may be performed by any one or more of: at least one conventional personal computer processor, workstation or other programmable device or computer or electronic computing device or processor, either general-purpose or specifically constructed, used for processing; a computer display screen and/or printer and/or speaker for displaying; machine-readable memory such as flash drives, optical disks, CDROMs, DVDs, BluRays, magnetic-optical discs or other discs; RAMs, ROMs, EPROMs, EEPROMs, magnetic or optical or other cards, for storing, and keyboard or mouse for accepting. Modules illustrated and described herein may include any one or combination or plurality of: a server, a data processor, a memory/computer storage, a communication interface (wireless (e.g. BLE) or wired (e.g. USB)), a computer program stored in memory/computer storage.

The term “process” as used above is intended to include any type of computation or manipulation or transformation of data represented as physical, e.g. electronic, phenomena which may occur or reside e.g. within registers and/or memories of at least one computer or processor. Use of nouns in singular form is not intended to be limiting; thus the term processor is intended to include a plurality of processing units which may be distributed or remote, the term server is intended to include plural typically interconnected modules running on plural respective servers, and so forth.

The above devices may communicate via any conventional wired or wireless digital communication means, e.g. via a wired or cellular telephone network or a computer network such as the Internet.

The apparatus of the present invention may include, according to certain embodiments of the invention, machine readable memory containing or otherwise storing a program of instructions which, when executed by the machine, implements all or any subset of the apparatus, methods, features and functionalities of the invention shown and described herein. Alternatively or in addition, the apparatus of the present invention may include, according to certain embodiments of the invention, a program as above which may be written in any conventional programming language, and optionally a machine for executing the program, such as but not limited to a general purpose computer which may optionally be configured or activated in accordance with the teachings of the present invention. Any of the teachings incorporated herein may, wherever suitable, operate on signals representative of physical objects or substances.

The embodiments referred to above, and other embodiments, are described in detail in the next section.

Any trademark occurring in the text or drawings is the property of its owner and occurs herein merely to explain or illustrate one example of how an embodiment of the invention may be implemented.

Unless stated otherwise, terms such as, “processing”, “computing”, “estimating”, “selecting”, “ranking”, “grading”, “calculating”, “determining”, “generating”, “reassessing”, “classifying”, “generating”, “producing”, “stereo-matching”, “registering”, “detecting”, “associating”, “superimposing”, “obtaining”, “providing”, “accessing”, “setting” or the like, refer to the action and/or processes of at least one computer/s or computing system/s, or processor/s or similar electronic computing device's or circuitry, that manipulate and/or transform data which may be represented as physical, such as electronic, quantities e.g. within the computing system's registers and/or memories, and/or may be provided on-the-fly, into other data which may be similarly represented as physical quantities within the computing system's memories, registers or other such information storage, transmission or display devices or may be provided to external factors e.g. via a suitable data network. The term “computer” should be broadly construed to cover any kind of electronic device with data processing capabilities, including, by way of non-limiting example, personal computers, servers, embedded cores, computing system, communication devices, processors (e.g. digital signal processor (DSP), microcontrollers, field programmable gate array (FPGA), application specific integrated circuit (ASIC), etc. and other electronic computing devices. Any reference to a computer, controller or processor is intended to include one or more hardware devices e.g. chips, which may be co-located or remote from one another. Any controller or processor may for example comprise at least one CPU, DSP, FPGA or ASIC, suitably configured in accordance with the logic and functionalities described herein.

Any feature or logic or functionality described herein may be implemented by processor/s or controller/s configured as per the described feature or logic or functionality, even if the processor's or controller/s are not specifically illustrated for simplicity. The controller or processor may be implemented in hardware, e.g., using one or more Application-Specific Integrated Circuits (ASICs) or Field-Programmable Gate Arrays (FPGAs), or may comprise a microprocessor that runs suitable software, or a combination of hardware and software elements.

The present invention may be described, merely for clarity, in terms of terminology specific to, or references to, particular programming languages, operating systems, browsers, system versions, individual products, protocols and the like. It will be appreciated that this terminology or such reference/s is intended to convey general principles of operation clearly and briefly, by way of example, and is not intended to limit the scope of the invention solely to a particular programming language, operating system, browser, system version, or individual product or protocol. Nonetheless, the disclosure of the standard or other professional literature defining the programming language, operating system, browser, system version, or individual product or protocol in question, is incorporated by reference herein in its entirety.

Elements separately listed herein need not be distinct components, and alternatively may be the same structure. A statement that an element or feature may exist is intended to include (a) embodiments in which the element or feature exists; (b) embodiments in which the element or feature does not exist; and (c) embodiments in which the element or feature exist selectably e.g. a user may configure or select whether the element or feature does or does not exist.

Any suitable input device, such as but not limited to a sensor, may be used to generate or otherwise provide information received by the apparatus and methods shown and described herein. Any suitable output device or display may be used to display or output information generated by the apparatus and methods shown and described herein. Any suitable processor/s may be employed to compute or generate or route, or otherwise manipulate or process information as described herein and/or to perform functionalities described herein and/or to implement any engine, interface or other system illustrated or described herein. Any suitable computerized data storage e.g. computer memory may be used to store information received by or generated by the systems shown and described herein. Functionalities shown and described herein may be divided between a server computer and a plurality of client computers. These or any other computerized components shown and described herein may communicate between themselves via a suitable computer network.

The system shown and described herein may include user interface/s e.g. as described herein which may, for example, include all or any subset of: an interactive voice response interface, automated response tool, speech-to-text transcription system, automated digital or electronic interface having interactive visual components, web portal, visual interface loaded as web page/s or screen/s from server/s via communication network/s to a web browser or other application downloaded onto a user's device, automated speech-to-text conversion tool, including a front-end interface portion thereof and back-end logic interacting therewith. Thus the term user interface or “UI” as used herein includes also the underlying logic which controls the data presented to the user e.g. by the system display and receives and processes and/or provides to other modules herein, data entered by a user e.g. using her or his workstation/device.

### BRIEF DESCRIPTION OF THE DRAWINGS

Certain embodiments of the present invention are illustrated in the following drawings; in the block diagrams, arrows between modules may be implemented as APIs and any suitable technology may be used for interconnecting functional components or modules illustrated herein in a suitable sequence or order e.g. via a suitable API/Interface. For example, state of the art tools may be employed, such as but not limited to Apache Thrift and Avro which provide remote call support. Or, a standard communication protocol may be employed, such as but not limited to HTTP or MQTT, and may be combined with a standard data format, such as but not limited to JSON or XML.

Methods and systems included in the scope of the present invention may include any subset or all of the functional blocks shown in the specifically illustrated implementations by way of example, in any suitable order e.g. as shown. Flows may include all or any subset of the illustrated operations, suitably ordered e.g. as shown. Tables herein may include all or any subset of the fields and/or records and/or cells and/or rows and/or columns described.

FIGS. 1-4 are simplified semi-pictorial semi-block diagram illustrations of 4 respective embodiments of the present invention.

Computational, functional or logical components described and illustrated herein can be implemented in various forms, for example, as hardware circuits such as but not limited to custom VLSI circuits or gate arrays or programmable hardware devices such as but not limited to FPGAs, or as software program code stored on at least one tangible or intangible computer readable medium and executable by at least one processor, or any suitable combination thereof. A specific functional component may be formed by one particular sequence of software code, or by a plurality of such, which collectively act or behave or act as described herein with reference to the functional component in question. For example, the component may be distributed over several code sequences such as but not limited to objects, procedures, functions, routines and programs and may originate from several computer files which typically operate synergistically.

Each functionality or method herein may be implemented in software (e.g. for execution on suitable processing hardware such as a microprocessor or digital signal processor), firmware, hardware (using any conventional hardware technology such as Integrated Circuit technology) or any combination thereof.

Functionality or operations stipulated as being software-implemented may alternatively be wholly or fully implemented by an equivalent hardware or firmware module, and vice-versa. Firmware implementing functionality described herein, if provided, may be held in any suitable memory device and a suitable processing unit (aka processor) may be configured for executing firmware code. Alternatively, certain embodiments described herein may be implemented partly or exclusively in hardware, in which case all or any subset of the variables, parameters, and computations described herein may be in hardware.

Any module or functionality described herein may comprise a suitably configured hardware component or circuitry. Alternatively or in addition, modules or functionality described herein may be performed by a general purpose computer or more generally by a suitable microprocessor, configured in accordance with: methods shown and described herein, or any suitable subset, in any suitable order, of the operations included in such methods, or in accordance with methods known in the art.

Any logical functionality described herein may be implemented as a real time application, if and as appropriate, and which may employ any suitable architectural option such as but not limited to FPGA, ASIC or DSP or any suitable combination thereof.

Any hardware component mentioned herein may in fact include either one or more hardware devices e.g. chips, which may be co-located or remote from one another. Any method described herein is intended to include, within the scope of the embodiments of the present invention, also any software or computer program performing all or any subset of the method's operations, including a mobile application, platform or operating system e.g, as stored in a medium, as well as combining the computer program with a hardware device to perform all or any subset of the operations of the method.

Data can be stored on one or more tangible or intangible computer readable media stored at one or more different locations, different network nodes, or different storage devices at a single node or location.

It is appreciated that any computer data storage technology, including any type of storage or memory and any type of computer components and recording media that retain digital data used for computing for an interval of time, and any type of information retention technology, may be used to store the various data provided and employed herein. Suitable computer data storage or information retention apparatus may include apparatus which is primary, secondary, tertiary or off-line; which is of any type or level or amount or category of volatility, differentiation, mutability, accessibility, addressability, capacity, performance and energy use; and which is based on any suitable technologies such as semiconductor, magnetic, optical, paper and others.

### DETAILED DESCRIPTION OF CERTAIN EMBODIMENTS

A system aka reference system for measuring a state of health of subject to be monitored, is provided. The system includes three sensors ((a) radar, (b) thermal camera, and (c) wide angle camera. or any subset thereof).

A system of systems may be used for highly increased reliability and accuracy of a measurement setup.

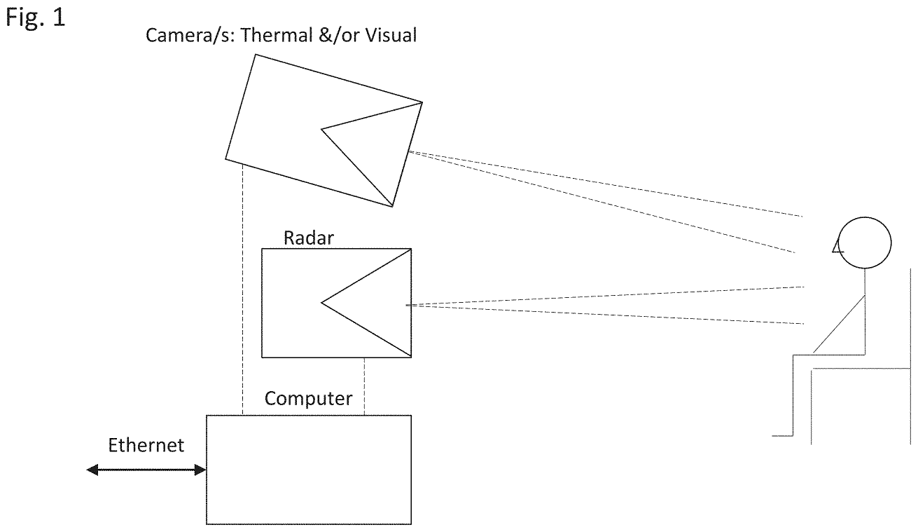

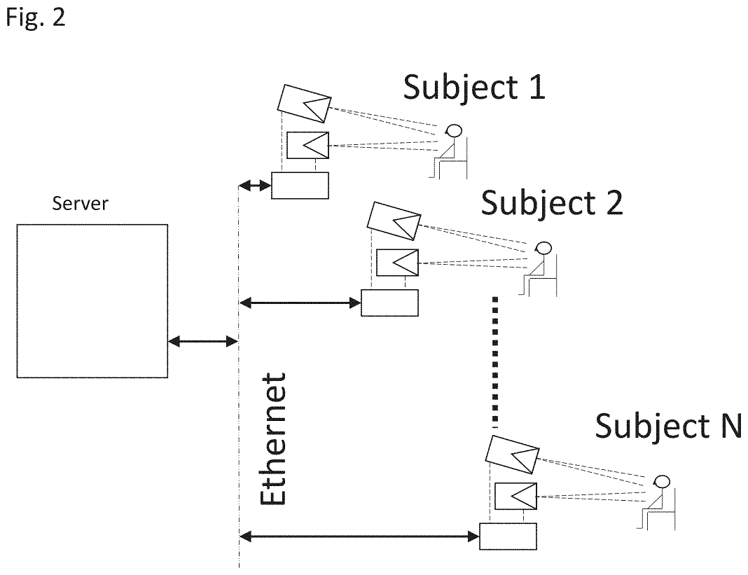

Example embodiments are shown in FIGS. 1-4. FIG. 1 shows a single system collecting data from a single subject, using a radar sensor and/or camera/s which may include thermal and/or visible light camera/s. FIG. 2 shows a system of systems, including plural subsystems, each collecting data from a single subject. All subsystems feed into a single server which may be used for registration and/or statistics, and/or for updating algorithms.

Computational power may be divided between:

a. a system local computer that may include real-time assessment algorithms e.g. as described herein and/or comparison to measurement standards if such exist, and/or system activation and/or manipulation, and/or Graphical User Interface, and

b. a server which can be either local or remote, to which all systems may be connected, via Ethernet, Wi-Fi or any other communication protocol. The server may, for example, be used to store and register vital signs data, according to subject identification and parameters, measurement time, location, and transfer the vital signs data to other computerized systems used by other human operators such as but not limited to medical personnel. Alternatively or in addition, the server may activate post processing of the data for determining success rates of plural algorithms used per scenario, e.g, as described herein, and may then implement changes in the algorithms and/or define new criteria for the use of specific algorithms e.g. according to specific scenarios or subjects' parameters.

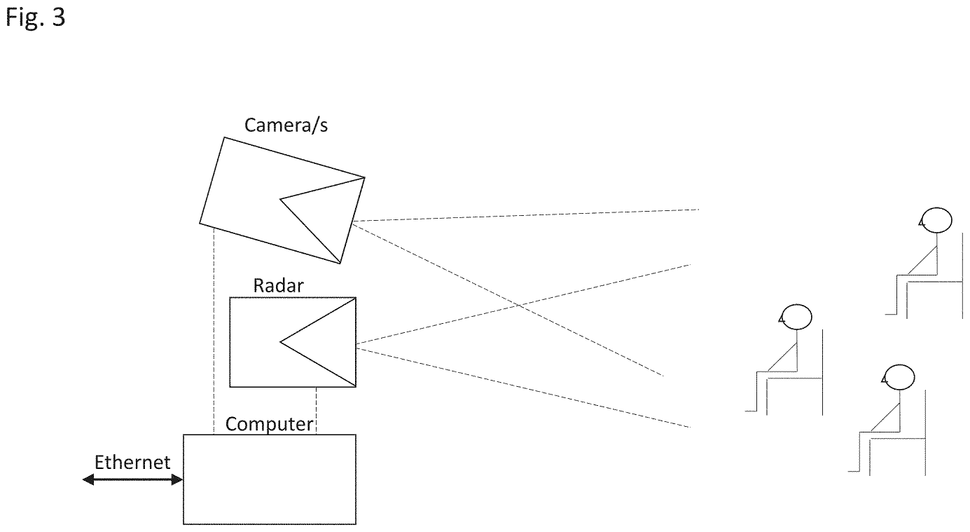

FIG. 3 shows a single system collecting data from plural subjects, using a radar sensor and/or camera/s which may include thermal and/or visible light camera/s.

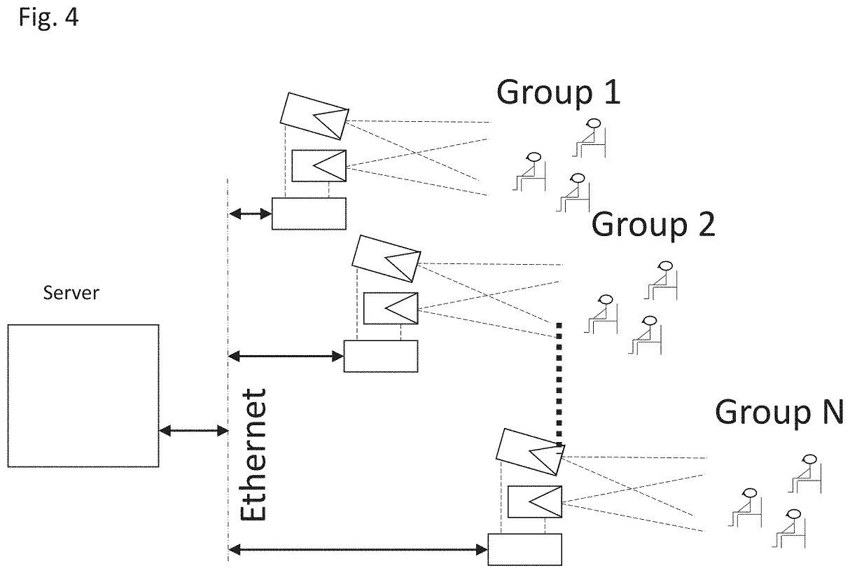

FIG. 4 shows a system of systems, including plural subsystems, each collecting data from plural subjects. Groups 1, 2, . . . of subjects served by subsystems 1, 2, . . . respectively need not be equal in size; for simplicity only, each group of subjects is shown in included 3 subjects. All subsystems feed into a single server, as in FIG. 2.

Instructions herein re placement, calibration and measurement volume typically apply to each and every sub system included.

All subsystems or sensors synchronized. Data recordings, typically of all systems, are typically directed to a single computer that is used to synchronize and control all the subsystems.

Typically, the radar is directed to the back of the monitored subjects, whereas the thermal and/or wide angle cameras are directed at the subjects' faces or subjects' profiles.

For a non-cooperative scenario, there is no limitation over the positions of the monitored subjects, other than their presence within the predefined volume (typically at minimal to maximal stated distances from the sensors, typically with a predefined angular sector.

The measurement setup includes a control post that includes a central computer, and a measurement volume defined by a minimal and maximal range, e.g. at a predefined angular sector, to the relevant sensors.

Each one of the sensors of the system is functional, calibrated, and deployed at a suitable distance e.g. 3 meters or more, from a volume to be monitored, e.g. room or passageway, in or through which the subject may pass.

Typically, a measurement volume without, or with as few as possible, stationary reflecting objects and heat emitting appliances, is chosen.

The measurement volume may be either controlled (e.g. in a hospital admittance room), or un-controlled (in which monitored subjects are unsupervised, thus are moving freely e.g. at an entrance passage to hospital, airport).

Each volume is typically chosen to comply with predetermined minimal and maximal ranges, as well as angular azimuth predefined sector. Thus, typically, even an uncontrolled volume has known dimensions which typically comply with volumetric stated definitions.

In a controlled volume, the monitored subjects may be situated in predetermined positions to ensure reliable measurement.

The physical separation between monitored subjects will allow to separate them by the radar range and azimuth resolution. Thus measurement in a control led environment prevents reliability degradation of monitored parameters.

An uncontrolled volume typically involves activating Artificial Intelligence algorithms for separating relevant from irrelevant measured data.

An uncontrolled volume may be arranged to facilitate the measurement process e.g. a preset walking path dictating single person passage or singulation of monitored persons, or movement in a single file within the measurement volume.

Typically, the use of a radar with the maximal EIRP (effective incident radiated power)—Radar EIRP is a sum of the transmitted power and the transmitting antenna gain within the limits of either FCC (26 dBm) or ETSI (20 dBm) standards allows the detection of heartbeat and respiration at distances of up to 50 m.

The transmit antenna gain is typically derived from the monitored angular volumetric section.

In a reference setup, an antenna with angular coverage of 11°×80° and resulting gain of ˜12 dBi may be chosen, together with transmission power of 8 dBm or 14 dBm, according to compliance to a relevant standard for maximal RF transmitted radiation per frequency band e.g. FCC (for US) and ETSI (for EU).

Distances beyond that e.g. above 50 m, are possible, taking into account multi path and multi reflection from reflectors in the surrounding area. Typically, arranging the long-range measurement volume includes mitigating multi reflection and multipath effects e.g. by (a) clearing the volume from unnecessary reflecting objects, (b) using “diffraction fences” or RF radiation absorbing material surfaces at ranges and angles from which undesired reflections are expected.

The large maximal detection range, together with a minimal range cell of down to 15 cm, enables vital signs measurement of number of people, simultaneously, with minimal interference between them. The number of people may vary between a cooperative scenario, in which the subjects are stationary (say 15-20 subjects) and a non-cooperative one, in which the subjects are constantly moving (typically less e.g. 5-10 subjects).

In controlled measurements, the monitored subjects may be placed at different ranges, e.g. more than two range gates apart, and distributed among the different radar azimuth beams.

The monitored subjects in a controlled measurement may be placed in a geometry that prevents blockage of the line of sight between any of them and the radar. For example, the radar may be placed above the subjects, looking down at them.

In an uncontrolled environment, the chosen measurement volume may be chosen similarly, but varying multi reflections may exist and dictate a lower effective measurement time, causing some measurements to be invalid for analysis.

The result of the lower effective monitoring time might be a reduced measurement reliability.

The minimal range cell may be achieved using a large bandwidth mode, typically FMCW, e.g. 15 cm for a 1 GHz bandwidth, for instance.

A short range cell may increase the radar sensitivity to the monitored subject (involuntary) movement.

A large range cell may include reflections from various parts of the monitored subject's body.

A cooperative setup may include a stationary screen (either metallic or absorptive) to exclude the lower part of the body from the measurements.

The radar sensor typically operates in a multi-beam mode of operation, typically allowing continuous and optimal angular separation and accuracies, and typically applying angular guard mechanisms (e.g. distinguishing a directly received signal from a person, from an indirectly received signal). The angular resolution of the radar is typically dependent on the cross section of the radar and its operation frequency. For example, the radar beam width may be 21 degrees for a 10 cm wide radar antenna at X-band frequency. High angular resolution, together with range cell differentiation, allows mitigating interference influences, while continuous measurement focus is enabled for subjects.

The multi beam mode typically allows simultaneous measurement of the entire volume at a very high update rate which lowers the influence of sporadic events (noise or interference, and sporadic movement of the monitored subject).

Simultaneous measurement at different angles allows creation of guard mechanisms for discarding interferences caused by reflections present at angle/s other than the angle in which a monitored subject is present.

The field of view is mapped. The outcome of the mapping process typically comprises the exact position and angle of all monitored subjects.

The mapping of the field of view is achieved by a software process of grid. mapping performed in order to locate the subjects to be monitored. The grid may be three dimensional e.g. in range, azimuth, and Doppler or phase change.

Once subjects are detected, subjects continue to be tracked throughout the measurement process.

When monitored subjects are mapped, a continuous phase measurement is performed for each subject at the specific range cell and azimuth beam. The measurement time (window) is typically larger than the period of the measured signal e.g. heartbeat or respiration, and typically takes into account relevant parameters such as, say, heartbeat of 55-130 per minute and respiration rates of 5-30 per minute for healthy subjects, excluding infants of age up to 1-year. Normal time measurement windows may, for example, be 5-10 s for respiration measurement, and 2-5 s for heartbeat measurement. The windows may vary according to the required measurements, and the measurements' quality and reliability.

The measured phase data is typically filtered in the sense of external noise or subject's movement removal, as well as singular events that are not related to heartbeat or respiration. Singular events' removal may be performed e.g. statistically and/or in conjunction with a Hidden Markov Model (HMM), digital filtering, Empirical Mode Decomposition (EMD), use of the Wavelet transform, or all of these algorithms in parallel. Weighting of these algorithms, and/or the choice of the most appropriate algorithms, may be performed by an Artificial Intelligence module which typically has the benefit of training data with known outputs from each of the various algorithms and known medical outcomes e.g. as stipulated by a known expert e.g. human medical expert.

Reduction of external or internal interferences received-power may be achieved by modifying the radar range cell, for instance from 15-60 cm in a quasi-arbitrary manner, e.g. by changing the FMCW (say) transmitted bandwidth, say from 1000-250 MHz.

Even though the range cell is modified, the Radar may still keep track of the monitored subjects, since the modification is predetermined.

Received signals may be compensated according to the specific range cell.

The measured phase may be re-filtered, e.g. by any method/s previously mentioned, for decoupling of heartbeat-respiration signals, and/or further de-coupling between different subjects, if required.

Heartbeat and respiration data may be computed and/or presented as averaged or high likelihood values, typically together with traces of real-time signals versus time, typically updating constantly, while interference related abnormalities are typically omitted.

Measurement time may span between 10-30 s, depending on the measurement scenario. This long measurement time may be used for additional filtering of external or internal interferences and/or ambient noise and/or subject's movement, whether involuntary (e.g. in case of a cooperative measurement), or voluntary (in case of an un-cooperative measurement).

A calibrated thermal camera may be used for all or any subset of (a) facial temperature monitoring, (b) respiration rate monitoring, (c) heart rate monitoring, and (d) lung tidal-volume or vital capacity estimation.

Respiration rate and heartbeat measured by the thermal camera may be used to corroborate the radar readings.

The thermal camera typically differentiates between distinct facial temperatures (which may be calibrated by black body apparatus).

While pointed at an angle to the face which allows the thermal camera to observe the monitored subject's nostrils and mouth, the thermal camera typically determines the temperature limited boundaries of the nostrils and the subject's mouth.

Knowing the exact distance to the subject, and the pixel matrix of the camera for a given angular coverage, allows dimensions of the mouth and nostrils' cross sections to be computed.

For a cooperative measurement—the distance to monitored subjects is known. Even for a non-cooperative measurement—the radar may exactly measure the distances to the monitored subjects.

When air is inhaled or exhaled either through the nose or through the mouth, the temperatures of the following face parts change: (a) nose, (b) lips, and facial surface between the upper lip and the nose.

The camera may detect air flow in and out of the nose and mouth e.g. by detecting and analyzing the temperature changes.

The exhaled air is warmer than the inhaled air, so, during inhalation, the relevant facial part gets cooler, or no warmer, over the time span of the inhalation, and during exhalation, the relevant facial parts get wanner over the time span of the exhalation.

By knowing the inhalation and exhalation relevant cross sections (nostrils, mouth, or both), and the air flow rate, picked up by the thermal camera—the lung tidal volume may be computed in an uncooperative measurement, and the lung vital capacity can be estimated at a cooperative measurement.

Air volume may be derived from the “air flux”, which may be measured in cubic meters per second, and the time span of the flow, which may be measured in seconds. Knowing the cross section of the flow (right nostril, left nostril, mouth, or their combination), measured in meters squared, and multiplying this cross section by the air velocity, measured in meters per second, yields the “air flux”.

The air flow may, for example, be assumed to be constant (transient time of the inhalation or exhalation beginning and end are negligible). Air temperature may be assumed to be constant throughout the process (either through inhalation or exhalation). These assumptions allow the inhaled or exhaled air volumes to be estimated, according to certain embodiments. The subtraction of estimated inhaled air volume and exhaled air volume, at rest, is relative to lung “tidal volume”.

If a person is asked to inhale deeply after the end of a normal inhalation at rest—the excess inhalation is relative to the “inspiratory reserve volume”. If a person is asked to exhale deeply after the end of a normal exhalation at rest—the excess exhalation is relative to the “expiratory reserve volume”. If a person is asked to inhale deeply and then exhale to the maximum, the difference in volumes will be the lungs' “vital capacity”.

According to certain embodiments, reserve volume of the lungs may be estimated and combined with at least vital capacity to yield Lung Capacity. For a non-cooperative measurement, typically, only the tidal volume is computed. However, for a cooperative measurement, in which a subject is asked to inhale and exhale deeply, within the test time-frame, the lung capacity may be extracted.

The respiration-period data may be used to corroborate the radar measurement and/or to increase the radar measurement's reliability and/or accuracy, e.g. by use of Artificial Intelligence.

Radar readings are used to measure respiration. Typically, plural algorithms are used for this purpose due to variability between monitored subjects differing in gender, age, height, or physical condition. Each algorithm produces somewhat different results. The AI module may be configured for assigning weights to each algorithm to reflect that algorithm's abilities in different scenarios, using a database accumulated over a long period of time. The measurement of the thermal camera may be used as an independent parameter to corroborate the result. Since the measurement is performed using a sensor which is “orthogonal” (does not have some of the limitations of the primary sensor such as multipath, multi reflections or RF coupling between received signals), this may be used to improve the learning process of the AI module.

Heart rate measurement by a thermal camera may be provided e.g. by monitoring the Carotid artery blood flow. This heart rate measurement may be used to corroborate the heart rate measured by the radar, e.g. as described above for respiratory rate assessment.

The thermal camera is pointed at one of the Carotid arteries (e.g. sides of the monitored subject's neck).

Blood flow through these arteries changes the surface temperature of the neck. These heat changes may be picked up by the thermal camera. Since blood through these arteries pulsates at the heart rate, the heartbeat rate may be estimated by the thermal camera by computing the rate of periodic heat change.

A thermal camera position may be chosen so that the cameras field of view includes both the subject's nostrils and mouth, as well as the monitored subject's neck.

The thermal camera parameters are typically determined according to the measurement scenario, e.g. taking into account all or any subset of (a) maximal measurement distance. (b) angular coverage, (d) number of pixels at the measurement area (subjects' faces).

In order to increase the temperature measurement accuracy of the thermal imaging, a dual wavelength thermal camera may be used.

The thermal camera is typically calibrated before use, typically using a “black body” apparatus.

An additional wide-angle (typically visible light) camera may be used to determine the movement type (if any) of a subject, in order to improve the measurement process quality. Typically, measurement anomalies associated with interferences and/or movement are identified and removed computationally. For example, the visible light wide-angle camera may pick up the movement of the person. Although in a controlled measurement, the monitored persons may be instructed not to move during the measurement, in practice some movement may be expected. The wide-angle camera picks up the movement and gives the movements a “time tag” or timestamp. The radar may use this timestamp to locate and remove timestamped portions within the measured data, thereby to retain, for analysis, only received data that is related to respiration and heartbeat.

In an uncontrolled measurement the wide-angle camera may be used for assigning measurements to persons. Since a customary camera AI module may be integrated for identifying monitored persons, the wide-angle camera picks up all moving persons within the monitored volume and tracks these persons. In this manner, measured vital signs may be assigned to relevant persons in an uncontrolled volume.

More generally, it is appreciated that heartbeat and respiration period measurement quality may be adversely affected by the monitored subject's movement, which may express itself in a received “phase change” that influences accuracy of computation/s of respiration or heartbeat period. Therefore, a process for removing phase changes which result from causes other than the heart and lungs of a person, hence are undesired or artefactual. A computational model may be employed or a camera e.g. wide angle camera observing the field of view may add this data. According to certain embodiments, once a scenario is learned, mathematical models which prove successful may be given a higher weighting, regarding other models less successful in determining correctly measured parameters.

While the vital signs of a few subjects are measured, the system may follow the subjects by the specific vital signs and their variation over time. This process may serve as a “Target Recognition” process for a scenario of multiple measured subjects. A wide-angle camera may contribute to the reliability of this process e.g. as described above.

Within the frame of a standalone system, Artificial Intelligence software may be used to assess measurements from all system sensors (e.g. radar and/or thermal camera and/or wide angle camera) and to generate a time coherent presentation of relevant parameters e,g. heartbeat and/or respiration and/or lung “tidal volume”.

The vital signs of all measurements are typically processed and stored.

Criteria for normal or abnormal parameters may be implemented within the system in order to warn or give notice in case of required attention.

Stored data may be transmitted to a data cloud.

Data within the cloud from plural systems may be analyzed, and processing algorithms used by the plural systems may be updated accordingly. The data may be used for further improvement of the Artificial Intelligence algorithms for each system.

Processing software may be uploaded from the cloud to each system.

Artificial Intelligence implemented within the system may include “machine learning” and/or “deep learning”. The machine learning module typically imposes or applies a (typically learned, on training data) set of rules over measured data for determining a best evaluation of vital signs monitored. In the “system of systems” described herein, plural systems are monitoring subjects from different distances and angles: each system senses something else. The AI module is configured to use synchronized data from the plural systems to get a clear monitoring “picture” of each and every subject. The AI module is previously trained and corroborated using a suitable pre gathered database. For example, if the system is being used to detect a virus such as Covid-19, the database may include persons known to be infected by the virus as well as persons known not to be infected, considering the predetermined measurement scenarios. Within the system, many algorithms are typically used in parallel, for the radar and/or for the thermal camera, e.g. because of the large variability of subjects (gender, build, height, and/or inherent physical conditions including long-term illnesses). Known physical conditions information may be supplied as external data not sensed by the system. A relevant pre-trained AI module may choose an algorithm or processing which is relevant for each given scenario.

The use of a wide angle camera may include conventional face recognition software such that a suitably configured AI module allows the system to attach or associate measured data to a specific person. For a closed environment such as a hospital ward, the system may monitor a given subject repeatedly and automatically, and accurately assign measurement to specific subjects, e.g. using conventional Automatic Target Recognition techniques.

While the system collects data, the system may indicate and alert of changes within the data, according to predetermined thresholds.

The AI module may be configured for choosing the appropriate processing. Alternatively or in addition, the AI module may automatically and actively change the processing modules themselves through long term “learning” and training. An AI module may be used to determine head and/or face position relative to the thermal camera. The black body apparatus may be identified by suitable software e.g. the same software module.

“Deep learning” typically includes using a neural network that is trained, given a sufficient database. While a sufficient database exists for all relevant scenarios described above, the system may be trained using “pre tagged” or labelled data of respiratory rate and/or heartbeat and/or facial temperature and/or monitored subjects' movement to determine the subjects' accurate vital signs (e.g. all or any subset of pulse rate, body or facial temperature, respiration rate, tidal volume), and whether the vital signs comply with, or fall within, a certain predefined span, as opposed to crossing certain typically predefined thresholds or limits.

Typically, the thermal camera includes plural camera units each responsive within a certain range of IR (InfraRed) wavelengths; together, the plural camera units cover required ranges.

Typically, the camera or an external hardware processor is operative to estimate nostril and/or mouth cross-section, and/or velocity of air being exhaled and/or inhaled. This data is typically translated to lung “tidal volume” using any suitable Artificial Intelligence (AI) algorithm e.g. as described above. In a controlled measurement where a person may be instructed how and when to breathe, more parameters reflecting the lungs may be extracted using a relevant AI module.

Typically, the system has (relatively) long range operation, yet is configured to overcome difficulties which occur in long-range operation such as radar multipath and multi reflection.

Respiration data typically includes data characterizing inhalation and exhalation of air, and the total time period thereof.

Lung “tidal volume” may be derived from respiration data estimated by IR cameras.

It is appreciated that all or any algorithms or computations described herein may be replaced entirely or in part, or augmented, by machine learning applied directly to raw data generated, e.g. as described herein, by the sensors, or to data. derived from such raw data.

Any suitable radar calibration techniques may be employed. Typically, the radar sensor comprises a CW (Continuous Wave) radar, although pulsed radar may be used.

The thermal camera may be calibrated using a “black body” apparatus that allows exact temperature calibration of the camera. Black body radiation refers to the thermal radiation of a body in equilibrium, for which emitted wavelength power density is dependent only on the body's temperature. The thermal camera typically operates at a specific wavelength which is known. A “black body apparatus” may be deployed which is known to be in equilibrium at a pre known temperature, and to emit power, at a specific wavelength, relative to the body's temperature. A setup process may deploy one or more black body apparatus within the thermal camera field of view. The thermal camera measures the temperature of subjects and simultaneously picks up the temperature of the black body apparatus. Since the exact temperature of the apparatus is pre known, all temperature readouts of the thermal camera may thereby be accurately calibrated online.

Typically, the radar comprises a multi beam radar (staring radar) or has a multi-beam radar mode which may use a conventional algorithm such as that used in acoustical devices and/or ground surveillance radars.

Unlike mechanically or electronically steered radars, which point a beam to a specific direction (while ignoring other directions at the same moment) and then, steering the field of view at a certain rate, a staring radar looks at all the field of view at all times and updates it simultaneously at a high rate. This facilitates excellent detection probability and very low false alarms for the use-case described herein, whereas normally, short distance radars do not use such a method which is less cost-effective compared to other modes of operation.

The staring mode is thus advantageous in covering a large field of view simultaneously, without time gaps caused by steering the radar beam, and may characterize interferences from different directions, and disqualify or discount them.

Stare-While-Scan technology may be employed which typically uses Digital Beam Forming (DBF) on the receive side, and a wide beam on the transmit side illuminating the entire scanned sector simultaneously, and replacing the mechanical or electronic steering mode. The continuous radar coverage allows very high update rates, resulting in low false alarm rates and high detection probability. By taking advantage of high-speed analog-to-digital converters to digitally synthesize the antenna beams, higher reliability is achieved in comparison to radars lacking this technology, which permits the radar to stare over the full 90-100° field-of-view and create multiple simultaneous beams that provide target location information at a high update rate. For example, the radar system aka radar sensor may update an entire 90°-360° field-of-view a few times per second.

The advantage of a high update rate is that the tracker maintains a more accurate target track, even for partially obscured targets that may move in and out of view, and maintains a continuous track for multiple targets that cross over, avoiding confusion or loss of targets. At slower update rates, a target that may be obscured for several seconds, and then visible again for a brief period, may not be updated or associated with clutter or a different target if the radar scanning update is too slow.

The radar may include a single transmit module and plural e.g. 32 receive modules, for instance, typically arranged as a linear or 2D array and providing multiple receiving beams. All receiving elements may be digitally processed to generate multiple separate, but simultaneous beams, yielding accuracy of detection and minimization of false alarms that maximises single or multiple target detection.

Typically, continuous transmission, across an entire area of interest, is provided at the same time. Also, continuous reception, across an entire area of interest, is provided at the same time. This allows the system to achieve higher integration times which translates into lower power consumption. Typically, a single wide transmission beam covers a wide area of interest (e.g. 100°). The plural receiving elements with the (optional) advanced multi-beam processing technique create ‘virtual’ receiving beams over all areas of interest simultaneously.

Staring radars, which lack scanning, are not subject to the inherent trade-off of scanning radars, thus may provide all of the following using the same mode:
1. Low MDV (minimal detectable velocity) or slow phase change
2. High update rate
3. High azimuth accuracy
4. 3D target tracking using high range, azimuth and Doppler or phase change separation.

Typically, a database is collected which stores all or any subset of (a) facial temperature, (b) respiration rate, and (c) lung tidal volume estimation in association with independent diagnoses of the same subjects e.g. Covid-19 diagnoses which may be binary (ill/healthy) or may quantify probability of infection, given the data collected from certain subjects.

Typically, an operator's display is provided which may present any outputs described herein.

Alternatively or in addition, outputs described herein may be fed e.g. via suitable APIs (application program interface) to other computerized systems.

Features of the present invention, including method steps, which are described in the context of separate embodiments, may also be provided in combination in a single embodiment. Conversely, features of the invention, which are described for brevity in the context of a single embodiment or in a certain order, may be provided separately, or in any suitable sub-combination, or in a different order. No parameter or specific value herein is intended to be limiting; instead these are merely exemplary in order to illustrate one possible useful value.

Advantages of certain embodiments include all or any subset of: a system of systems (e.g. radar and at least two cameras) which yields improved reliability and accurate monitoring; for example, an obscured line of sight to a monitored patient may prevent one of the systems from adequately picking up a received signal in a clear manner, whereas another of the systems does pick up the signal, yielding a combined, coherent presentation of the vital signs over time;

both indoor and outdoor usage,

quick and simple permanent, or even temporary deployment, even in a vehicle;

operator/s which need not be in the same room as monitored subjects and may be distanced therefrom to avoid contagion of operators by monitored subjects;

measurement of king tidal-volume by comparing differences between inhaled and exhaled air temperatures, velocities, and mouth and/or nostrils cross sections (as opposed to measuring volume of air exhaled by the monitored subject by a Spirometer);

reduced influence of interference, due to plural sensors which have different vulnerabilities to interference;

ability of one sensor to measure parameters that another (e.g. the radar) cannot measure, e.g. that a thermal camera may measure;
temperature (such as facial), with very high accuracy, especially if plural different wavelengths are employed; and/or
lung tidal volume as described herein.

Respiration data is used to corroborate the radar measurement and increase its reliability and accuracy, e.g. by use of Artificial Intelligence.

Very long distance measurement is possible, e.g. a subject may be monitored from 10 or 20 or 30 or 40 or 50 meters away.

A radar system aka radar sensor (such as ELT's ELM-2112 or ELM-2114) typically having a low false alarm rate may be achieved by all or any subset of: high update rate, spatial resolution, and multi beam algorithms.

It is appreciated that any radar system, typically having a multi-beam mode, and/or a FMCW mode which may be combined with the multi-beam mode if provided, may be used for any of the embodiments shown and described herein. The radar may, for example, be characterized by all or any subset of the following:
1. Radar uses an ISM (Industrial, Scientific, Medical) frequency band (example: 24.0-24.25 GHz) that needs no frequency certification. It is appreciated that determination of which frequency band is ISM may vary between continents and/or countries; the relevant definitions are available online, for instance, FCC in the US, and ETSI in Europe.
2. Radar transmitted power level is controlled and/or fixed according to FFC (USA) or ETSI (Europe) standard requirements.
Example: maximal ERP (Effective Radiated Power) is 20 dBm according to the ETSI standard and 26 dBm according to the FCC standard. The radar system typically comprises, also, with regulation-defined levels of spurious emission, frequency harmonics, and antenna's side lobe level.
3. Received signals discriminated in one or more axes e.g. time and/or range and/or angle and/or velocity, and/or power axes, so as to facilitate focusing on relevant data, and disqualifying nuisance data.

Nuisance data may, for example, be caused by: electromagnetic energy emitters in the vicinity of the system, and/or by internal noise within the radar caused by the active RF components of the radar circuitry, and/or by moving elements within the radar field of view (e.g. fans and/or vibrating air-conditioning outlets and/or moving foliage observed through windows, and/or moving persons, other than the person of interest, within the radar field of view, etc.). Nuisance data for the thermal camera may be any heat emanating object within the thermal camera field of view other than the subjects and the black body apparatus.
4. Multi-beam mode, if provided, enables using software defined spatial real-time “Guard mechanisms” for disqualifying nuisance data false detections.
5. Radar may include Multiple-input multiple-output (MIMO) setup in Azimuth (and/or elevation, depending on the installation) to increase angular accuracy of measured data.
6. Multi-beam mode, if provided, may be combined with a waveform e.g. n FMCW waveform which lengthens the coherent integration time for measured data, relative to a radar system where there is no multi-beam mode, or where the multi-beam mode is not combined with an FMCW waveform. This lengthened coherent integration time reduces thermal noise power within measurements (aka processing gain enhancement), and therefore allows powers to be received or sensed by the system which are extremely low relative to the powers that could be received if thermal noise power were not so reduced. This allows the system to accurately sense heart and lung movements, which may be associated with extremely low powers, which may, in some use-cases, be −130 dBm or lower.
7. When the radar operates in FMCW mode, if such is provided, all or any subset of the parameters of the FMCW waveform can be changed from one integration period to another. This radar system property may be used to reduce probabilities of interferences, whether internal or external, being processed as relevant signals. The FMCW (say) radar parameters may include (a) scanned frequency bandwidth and/or (b) scan-rate and/or (c) coherent integration time and/or (d) data sampling rate. Real reflection can be distinguished from part of the nuisance data e.g. by changing one of these parameters, or all of them, sequentially or otherwise (or in a semi arbitrary or random manner, for instance).

It is appreciated that various sets of algorithms may be used by the system for various purposes respectively. For each purpose, a set of plural algorithms rather than a single algorithm may be provided, e.g. because of the large variability between subjects.

For example, as described, radar readings may be used to measure respiration. Typically, a set of plural algorithms is available for this purpose e.g. due to variability between monitored subjects differing in gender, age, height, or physical condition. Each algorithm produces somewhat different results. The AI module may be configured for assigning weights to each algorithm, using a database which the system builds up or accumulates over a large period of time, to reflect that algorithm's abilities in different scenarios.

It is appreciated that a measurement setup may be provided, in which subjects' vital signs are measured, both by the system of the present invention and by a standard e.g. a conventional contact based medical measurement standard. In this case, measurements by the system herein can be corroborated and stored for future reference, for each subject, or once per n subjects, such as once for every 100 subjects. Data thus collected can, over time, be used for algorithm improvement and for selecting relevant algorithms for specific scenarios and/or specific subjects' parameters.

Alternatively or in addition, a set of plural algorithms may be used in parallel, for the purpose of allowing the radar to generate outputs other than respiration. Radar algorithms provided by the system may be characterized by any of the following:
1. Filtering of nuisance data e.g. as described herein. Guard mechanisms may be provided for verifying that the received signals are legitimate radar reflections. For instance, a guard mechanism may be provided for differentiation of signals received through antennas' main lobes, and not through side lobes, taking into account that the optimized angular side lobes' levels of the radar are always lower than optimized angular main lobes' levels of the radar.
2. Measurement of heart rates may be dependent on variation or variability between subjects. The resulting heart rate, therefore, might vary significantly. Moreover, the received radar reflections might vary in power and phase over time, which makes it difficult to declare a true heart rate without a long measurement exceeding required reporting time, as in “contact measurements”. Different algorithms statistically yield better results of heart rate for specific subject types. Relevant algorithms employed might be “Hidden Markov Model” based, wavelet based transform, autocorrelation based transform, peak detection in amplitude over time, or in phase over time axis (or a combination of both). The algorithms are presented in literature, however, no attempt was evident to employ all of them, or a large part of them together, and decide which one is the relevant one.
3. The choice of a relevant algorithm can be made either by feature extraction (machine learning), or by using a neural network after a process of training. The advantage of using such a network here is that the amount of collected data is huge, meaning that the ability of the neural network to correlate the relevant process to a relevant subject will occur with a high probability, yielding an accurate measurement of the heart rate.

Alternatively or in addition, a set of plural algorithms may be used in parallel, for the thermal camera.

Alternatively or in addition, data regarding nostril and/or mouth cross-section, and/or regarding velocity of air being exhaled and/or inhaled, is collected, and a single Artificial Intelligence algorithm or plural such algorithms, may be used for the purpose of translating this data to lung tidal volume.

Alternatively or in addition, a single Artificial Intelligence algorithm or plural such algorithms, may be used for the purpose of separating relevant from irrelevant measured data, e.g. for uncontrolled volumes.

Alternatively or in addition, a single Artificial Intelligence algorithm or plural such algorithms, may be used for the purpose of singular events removal. Such removal may be performed e.g. statistically and/or in conjunction with a set of algorithms which may for example include Hidden Markov Model (HMM), and/or digital filtering, and/or Empirical Mode Decomposition (EMD), and/or use of the wavelet transform, or all of these algorithms in parallel. Weighting of these algorithms and/or the choice of the most appropriate algorithms may be performed by an Artificial Intelligence module which typically has the benefit of training data with known outputs from the various algorithms and known medical outcomes e.g. as stipulated by a known expert e.g. human medical expert.

A relevant pre-trained Artificial Intelligence aka AI module may choose an algorithm or processing, from among a set of plural available algorithms, which is relevant for each of several scenarios (e.g. each of various subjects from among a population of subjects characterized by variation between subjects, and/or each of various system tasks). It is appreciated that variability or variation between subjects as used in this document, including but not limited to variation in subjects' physical condition, may be operationalized by any suitable subject parameters (e.g. gender, and/or build (extremely thin to extremely obese), and/or height, and/or inherent physical conditions, including long term illnesses and/or weight, and/or physical shape and/or age). Known information specifying this variability may be supplied as external data and/or sensed by the system.

The Artificial Intelligence aka AI module may choose an algorithm or processing, from among a set of plural available algorithms by applying any suitable technology such as but not limited to (a) machine learning, and/or (b) neural networks, and/or (c) convolutional neural networks, to tagged data available to the system. For example, if the system task is to screen a stream of incoming subjects for possible Covid-19 infected subjects, tagged data may be available to the system which defines that certain subjects processed by the system in the past, did or did not turn out to be infected (as determined independently e.g. by conventional medical Covid-19 diagnosis).

Algorithms provided by the system may for example include all or any subset of the following:

FFT (Fast Fourier Transform) based algorithm: This algorithm uses the FFT transform, to transform time dependent amplitude data to spectral content of this data. For example, if relevant heart rate data and breath rate data are separated (different rates), and the rates are continuous and constant along the measurement time, FFT may be used. If heart rate data and breath rate data vary in time as well as in amplitude, and/or if in at least some cases, a low heart rate is combined with a high breath rate, algorithms other than FFT may be selected.

Wavelet based Algorithm: the wavelet transform expands the FFT transform by taking into account variations in the data along the axis to be transformed. For example, if the measured data (heart and breath rates) varies along the time axis, a wavelet transform may be selected, e.g. to present the changes as a basis for heart and breath rate determinations.

Peak detection based Algorithm: Heart and breath rates measurements can be recorded on a time axis in which case, typically, amplitude and/or phase peaks are counted along a. measurement period. It is appreciated that, for certain subjects, more than “one heart rate peak” can be measured within one heart cycle, due to more than one reflection per subject (such as chest movement combined with neck artery dynamics). This algorithm may be selected, or may not be selected, if, for example, involuntary movement of the subject has occurred or is likely; it is appreciated that this algorithm is sensitive to interferences and amplitude changes along the measurement period, which may be caused by involuntary subject movements.

Empirical Mode Decomposition (EMD) based algorithm: This typically comprises a heuristic process that tries to match a weighted function-base to a given data e.g, periodic heart and breath rate functions. The system may select this algorithm for certain scenarios and/or parameters' spans, and not for other scenarios and/or parameters' spans.

Hidden Markov Model (HMM) based algorithm: this may use a statistical Markov model for identifying either a “heartbeat” and “breath period”; or identifying the “heart and breath rates”. Typically, boundaries of the relevant parameters, as well as their variability in the relevant measurement period, are predefined.

It is appreciated that many variations on the embodiments shown and described herein are possible. Embodiments within the scope of the invention include, but are not limited to, all or any subset of the following:

Embodiment 1. A system for standoff measurement of medical data, characterizing the subject to be monitored, the system including at least one radar sensor and/or at least one electro-optical sensor, and wherein the medical data, typically including pulse and/or respiratory rate and/or temperature, may be measured remotely, thus reducing the risk of infection to medical personnel.

Embodiment 2. The system, either portable or at a fixed installation, according to any of the preceding embodiments, wherein the at least one radar sensor and at least one electro-optical sensor include all or any subset of an RF sensor, thermal camera/s, an optical sensor e.g. wide angle visible light camera; and wherein the system includes a hardware processor, which may be in a cloud, configured to process outputs of the above sensor/s. more generally, any processors referred to herein may be in a cloud and any computations described herein may be performed in a cloud.

Embodiment 3. The system according to any of the preceding embodiments, which includes an AI module, and wherein a system of systems is used to increase reliability and accuracy of measurement, and, typically, to allow future online and real-time modifications of existing algorithms, the system of systems including plural systems monitoring subjects from different distances and/or angles, and wherein the AI module is configured to use synchronized data from the plural systems to yield monitoring data regarding monitored subjects.

Embodiment 4. The system according to any of the preceding embodiments, wherein radar range and azimuth resolution are used when processing, to separate physically separated monitored subjects, as well as monitored subjects and nuisance detections, either thermal or moving elements in the radar field of view, thereby to yield measurement in a controlled environment which prevents reliability degradation of monitored parameters.

The subjects' physical separation typically enhances the ability of radar algorithms to differentiate computationally between these subjects. Since measurement time is typically limited, and/or hardware in the system is limited by performance as well as price, measurements from different subjects might interfere, and/or physical effects such as multi-path, and multi-reflection might cause a mix-up of measurements, especially if the volume in which the measurements are taken includes many passive reflectors e.g. tables, chairs, beds, or any stationary large object within the radar field of view.

Embodiment 5. The system according to any of the preceding embodiments, wherein a detection range of 10 or 20 or 30 or 40 or 50 or therebetween or more meters is provided, together with a radar range cell of 15 or 25 or 35 or 45 or 55 or 66 cm, less than, or therebetween, thereby to enable vital signs measurement of plural monitored subjects, simultaneously, with minimal interference between them, as well as interferences or radar reflections not caused by subjects within the field of view.

Embodiment 6. The system according to any of the preceding embodiments, wherein the radar sensor has a multi-beam mode of operation which allows simultaneous measurement of an entire volume having an update rate, preferably a high update rate, thereby to lower influence of sporadic events such as noise, interference, and sporadic movement of monitored subject/s. Typically, the multi-beam mode of operation allows performing multiple, simultaneous and real-time processing algorithms, from which the best measurement result can be extracted by means of machine learning or any other Artificial Intelligence software module or inference process.

Embodiment 7. The system according to any of the preceding embodiments, wherein the system's field of view is mapped, using a grid mapping operation which generates position/s and angle/s of monitored subject/s.

Embodiment 8. The system according to any of the preceding embodiments, wherein the grid mapping operation is four dimensional in range, azimuth, elevation and Doppler or phase change, or is three dimensional in range, azimuth, and Doppler or phase change, or in range, elevation, and Doppler or phase change.

Embodiment 9. The system according to any of the preceding embodiments, wherein the radar sensor defines a radar range cell, and wherein the radar range cell is modified between plural possible ranges in a quasi-arbitrary manner, thereby to reduce external or internal interferences of received-power.

Embodiment 10. The system according to any of the preceding embodiments, wherein the radar range cell is modified by changing an FMCW (say) transmitted bandwidth, from one frequency-band to another within a range of possible frequency-bands.

Embodiment 11. The system according to any of the preceding embodiments, wherein outputs (aka radar readings) of the radar sensor are used to measure respiration and wherein plural algorithms are used for this purpose and an AI module assigns weights to each algorithm to reflect that algorithm's abilities in different scenarios, using an existing database built up by the system over time.

Embodiment 12. The system according to any of the preceding embodiments, wherein the system includes a thermal camera and wherein heart rate measurements are generated by the system including pointing the thermal camera at at least one Carotid artery of at least one monitored subject and, accordingly, monitoring monitored subjects' Carotid artery blood flow, since blood flow through Carotid arteries changes the surface temperature of the neck, these heat changes being picked up by the thermal camera, blood through the Carotid arteries pulsates at heart rate, and wherein the heartbeat rate is then estimated by computing the rate of periodic heat change.

Embodiment 13. The system according to any of the preceding embodiments, wherein a wide-angle and/or visible light camera is used to determine movement type, if any, of monitored subject/s and, wherein, accordingly, measurement process quality is improved by identifying and computationally removing measurement anomalies associated with interferences and/or movement.

Embodiment 14. The system according to any of the preceding embodiments, wherein Artificial Intelligence software is used to assess measurements from plural system sensors (radar and/or thermal camera and/or wide angle camera), and, typically, from plural simultaneous real-time measurement algorithms, and to generate a time coherent presentation of relevant parameters.

Embodiment 15. The system according to any of the preceding embodiments, wherein the Artificial Intelligence software includes machine learning functionality and/or a neural network, and/or an inference process, which applies, to measured. data, rules learned on training data, for best evaluating vital signs from the measured data.

Embodiment 16. The system according to any of the preceding embodiments, wherein the system is used to detect which monitored subjects have a diagnosis, according to preset conditions, and which do not, according to same or different preset conditions. The training data may include data from subjects known to have the diagnosis, and from other subjects known not to have the diagnosis.

Embodiment 17. The system according to any of the preceding embodiments, wherein, as the system collects data, the system alerts of changes within the data and the Artificial Intelligence software automatically changes processing modules accordingly.

Embodiment 18. The system according to any of the preceding embodiments, wherein the radar sensor has a staring mode, thereby to cover a field of view simultaneously, yielding continuous radar coverage, without time gaps caused by steering the radar beam, thereby to support high update rates resulting in low false alarm rate and/or high detection probability, relative to lower update rates, higher false alarm rates and/or lower detection probabilities of scanning radars, and/or thereby to provide low MDV (minimal detectable velocity) or slow phase change and/or high azimuth accuracy, relative to higher MDV (minimal detectable velocity) and/or lower azimuth accuracies of scanning radars.

The high range, azimuth, radial-velocity radar accuracies and/or resolutions allow a subject's exact position to be identified, and allow the subject's measurements to be separated from other measured subjects' data or from interferences within the field of view of the radar sensor, which, if not separated, may degrade radar measurements generated by the radar sensor relative to radar measurements generated by a radar sensor in which the subject's measurements are separated.

Embodiment 19. The system according to any of the preceding embodiments, wherein, using the staring mode, the system is configured to characterize interferences from different directions, and disqualify or discount them on that basis.

Embodiment 20. The system according to any of the preceding embodiments, wherein, using the staring mode, the system is configured for 3D target tracking using high range, azimuth and Doppler or phase change separation, relative to scanning radars.

Embodiment 21. The system according to any of the preceding embodiments, wherein the system includes a thermal camera, and wherein air flow rate is sensed by the thermal camera, and wherein lung capacity is computed using air flow rate and estimates, known to the system, of inhalation and/or exhalation cross sections of at least one of nose and mouth.

Embodiment 22. A system according to any of the preceding embodiments which includes a thermal camera sensitive to at least one wavelength, and wherein body temperature measurements are generated by: pointing the thermal camera toward at least one subject's body part, the body part typically including at least one of the subject's face, forehead, neck, palm of hand, thereby to generate a first reading; pointing the thermal camera to a calibrated temperature generator, such as a. black body element at the at least one wavelength, thereby to generate a second reading; and generating a body temperature estimate by comparing the first and second readings.

Embodiment 23. A system according to any of the preceding embodiments which includes a thermal camera which generates first heart rate measurements which are combined with second heart rate measurements provided by the radar sensor, thereby to provide an extremely accurate evaluation of heart rate and/or to remove at least one heart rate measurement artifact and/or at least one false heart rate measurement.

Embodiment 24. The system according to any of the preceding embodiments wherein respiration rate is measured by the thermal camera using the inhaled and exhaled air flows.

For instance, if the inhalation time is 2 seconds and the exhalation time plus the time before the next exhalation is 4 seconds, then the total respiration time-period is 6 seconds which may result in a respiration rate of 10 breaths per minute.

Embodiment 25. The system according to any of the preceding embodiments wherein the thermal camera generates first respiration rate measurements which are combined with second respiration rate measurements provided by the radar sensor, thereby to provide an extremely accurate evaluation of respiration rate and/or to disqualify at least one false measurement caused by subject motion and/or disqualify at least one false measurement caused by interference within the system's field of view.

Embodiment 26. A method for measuring medical data characterizing a subject to be monitored, the system including at least one radar sensor and at least one electro-optical sensor, and wherein the medical data, including pulse and/or respiratory rate and, optionally temperature of the subject to be monitored, are measured remotely, thereby providing standoff detection and reducing the risk of infection to medical personnel.

Embodiment 27. A computer program product, comprising a non-transitory tangible computer readable medium having computer readable program code embodied therein, the computer readable program code adapted to be executed to implement a method for measuring medical data characterizing a subject to be monitored, the system including at least one radar sensor and at least one electro-optical sensor, and wherein the medical data, including pulse and/or respiratory rate and, optionally temperature of the subject to be monitored, are measured remotely thereby providing standoff detection and reducing the risk of infection to medical personnel.

It is appreciated that terminology such as “mandatory”, “required”, “need” and “must” refer to implementation choices made within the context of a particular implementation or application described herewithin for clarity, and are not intended to be limiting, since, in an alternative implementation, the same elements might be defined as not mandatory and not required, or might even be eliminated altogether.

Components described herein as software may, alternatively, be implemented wholly or partly in hardware and/or firmware, if desired, using conventional techniques, and vice-versa. Each module or component or processor may be centralized in a single physical location or physical device, or distributed over several physical locations or physical devices or may be in a cloud.

Included in the scope of the present disclosure, inter alia, are electromagnetic signals in accordance with the description herein. These may carry computer-readable instructions for performing any or all of the operations of any of the methods shown and described herein, in any suitable order, including simultaneous performance of suitable groups of operations, as appropriate. Included in the scope of the present disclosure, inter alia, are machine-readable instructions for performing any or all of the operations of any of the methods shown and described herein, in any suitable order; program storage devices readable by machine, tangibly embodying a program of instructions executable by the machine to perform any or all of the operations of any of the methods shown and described herein, in any suitable order i.e. not necessarily as shown, including performing various operations in parallel or concurrently rather than sequentially as shown; a computer program product comprising a computer useable medium having computer readable program code, such as executable code, having embodied therein, and/or including computer readable program code for performing, any or all of the operations of any of the methods shown and described herein, in any suitable order; any technical effects brought about by any or all of the operations of any of the methods shown and described herein, when performed in any suitable order; any suitable apparatus or device or combination of such, programmed to perform, alone or in combination, any or all of the operations of any of the methods shown and described herein, in any suitable order; electronic devices each including at least one processor and/or cooperating input device and/or output device and operative to perform, e.g. in software, any operations shown and described herein; information storage devices or physical records, such as disks or hard drives, causing at least one computer or other device to be configured so as to carry out any or all of the operations of any of the methods shown and described herein, in any suitable order; at least one program pre-stored, e.g. in memory, or on an information network such as the Internet, before or after being downloaded, which embodies any or all of the operations of any of the methods shown and described herein, in any suitable order, and the method of uploading or downloading such, and a system including server/s and/or client/s for using such; at least one processor configured to perform any combination of the described operations or to execute any combination of the described modules; and hardware which performs any or all of the operations of any of the methods shown and described herein, in any suitable order, either alone or in conjunction with software. Any computer-readable or machine-readable media described herein is intended to include non-transitory computer- or machine-readable media.

Any computations or other forms of analysis described herein may be performed by a suitable computerized method. Any operation or functionality described herein may be wholly or partially computer-implemented e.g. by one or more processors. The invention shown and described herein may include (a) using a computerized method to identify a solution to any of the problems or for any of the objectives described herein, the solution optionally including at least one of a decision, an action, a product, a service or any other information described herein that impacts, in a positive manner, a problem or objectives described herein; and (b) outputting the solution.

The system may, if desired, be implemented as a network—e.g. web-based system employing software, computers, routers and telecommunications equipment, as appropriate.

Any suitable deployment may be employed to provide functionalities e.g. software functionalities shown and described herein. For example, a server may store certain applications, for download to clients, which are executed at the client side, the server side serving only as a storehouse. Any or all functionalities e.g. software functionalities shown and described herein, may be deployed in a cloud environment. Clients e.g. mobile communication devices such as smartphones may be operatively associated with, but external to the cloud.

The scope of the present invention is not limited to structures and functions specifically described herein and is also intended to include devices which have the capacity to yield a structure, or perform a function, described herein, such that even though users of the device may not use the capacity, they are, if they so desire, able to modify the device to obtain the structure or function.

Any “if-then” logic described herein is intended to include embodiments in which a processor is programmed to repeatedly determine whether condition x, which is sometimes true and sometimes false, is currently true or false, and to perform y each time x is determined to be true, thereby to yield a processor which performs y at least once, typically on an “if and only if” basis e.g. triggered only by determinations that x is true, and never by determinations that x is false.

Any determination of a state or condition described herein, and/or other data. generated herein, may be harnessed for any suitable technical effect. For example, the determination may be transmitted or fed to any suitable hardware, firmware or software module, which is known or which is described herein to have capabilities to perform a technical operation responsive to the state or condition. The technical operation may for example comprise changing the state or condition, or may more generally cause any outcome which is technically advantageous given the state or condition or data, and/or may prevent at least one outcome which is disadvantageous, given the state or condition or data. Alternatively or in addition, an alert may be provided to an appropriate human operator or to an appropriate external system.

Features of the present invention, including operation which are described in the context of separate embodiments, may also be provided in combination in a single embodiment. For example, a system embodiment is intended to include a corresponding process embodiment, and vice versa. Also, each system embodiment is intended to include a server-centered “view” or client centered “view”, or “view” from any other node of the system, of the entire functionality of the system, computer-readable medium, apparatus, including only those functionalities performed at that server or client or node. Features may also be combined with features known in the art and particularly, although not limited to, those described in the Background section or in publications mentioned therein.

Conversely, features of the invention, including operations, which are described for brevity in the context of a single embodiment or in a certain order, may be provided separately or in any suitable sub-combination, including with features known in the art (particularly although not limited to those described in the Background section or in publications mentioned therein) or in a different order. “e.g.” is used herein in the sense of a specific example: which is not intended to be limiting. Each method may comprise all or any subset of the operations illustrated or described, suitably ordered e.g. as illustrated or described herein.

Devices, apparatus or systems shown coupled in any of the drawings may in fact be integrated into a single platform in certain embodiments, or may be coupled via any appropriate wired or wireless coupling, such as but not limited to optical fiber, Ethernet, Wireless LAN, HomePNA, power line communication, cell phone, Smart Phone (e.g. iPhone), Tablet, Laptop, PDA, Blackberry GPRS, satellite including GPS, or other mobile delivery. It is appreciated that in the description and drawings shown and described herein, functionalities described or illustrated as systems and sub-units thereof can also be provided as methods and operations therewithin, and functionalities described or illustrated as methods and operations therewithin can also be provided as systems and sub-units thereof. The scale used to illustrate various elements in the drawings is merely exemplary and/or appropriate for clarity of presentation, and is not intended to be limiting.

Any suitable communication may be employed between separate units herein e.g, wired data communication and/or in short-range radio communication with sensors such as cameras e.g. via WiFi, Bluetooth or Zigbee.

It is appreciated that implementation via a cellular app as described herein is but an example, and instead, embodiments of the present invention may be implemented, say, as a smartphone SDK, as a hardware component, as an STK application, or as suitable combinations of any of the above.

Any processing functionality illustrated (or described herein) may be executed by any device having a processor, such as but not limited to a mobile telephone, set-top-box, TV, remote desktop computer, game console, tablet, mobile e.g. laptop or other computer terminal, embedded remote unit, which may either be networked itself (may itself be a node in a conventional communication network e.g.) or may be conventionally tethered to a networked device (to a device which is a node in a conventional communication network or is tethered directly or indirectly/ultimately to such a node).

Any operation or characteristic described herein may be performed by another actor outside the scope of the patent application, and the description is intended to include any apparatus, whether hardware, firmware or software, which is configured to perform, enable or facilitate that operation, or to enable, facilitate or provide that characteristic.

The terms processor or controller or module or logic as used herein are intended to include hardware such as computer microprocessors or hardware processors, which typically have digital memory and processing capacity, such as those available from, say Intel and Advanced Micro Devices (AMD). Any operation or functionality or computation or logic described herein may be implemented entirely or in any part on any suitable circuitry, including any such computer microprocessor/s, as well as in firmware or in hardware, or any combination thereof.

It is appreciated that elements illustrated in more than one drawings, and/or elements in the written description may still be combined into a single embodiment, except if otherwise specifically clarified herewithin. Any of the systems shown and described herein may be used to implement, or may be combined with, any of the operations or methods shown and described herein.

It is appreciated that any features, properties, logic, modules, blocks, operations or functionalities described herein, which are, for clarity, described in the context of separate embodiments, may also be provided in combination in a single embodiment, except where the specification or general knowledge specifically indicates that certain teachings are mutually contradictory and cannot be combined. Any of the systems shown and described herein may be used to implement or may be combined with, any of the operations or methods shown and described herein.

Conversely, any modules, blocks, operations or functionalities described herein, which are, for brevity, described in the context of a single embodiment, may also be provided separately, or in any suitable sub-combination, including with features known in the art. Each element e.g. operation described herein may have all characteristics and attributes described or illustrated herein, or, according to other embodiments, may have any subset of the characteristics or attributes described herein.

1. A system for measuring medical data characterizing a subject to be monitored, the system including at least one radar sensor and at least one electro-optical sensor, and wherein the medical data, including pulse and/or respiratory rate and, optionally temperature of the subject to be monitored, are measured remotely, thereby providing standoff detection and reducing the risk of infection to medical personnel.

2. The system according to claim 1 wherein the at least one radar sensor and at least one electro-optical sensor include all or any subset of an RF sensor, thermal cameras, an optical sensor e.g. wide angle visible light camera; and wherein the system includes a hardware processor configured to process outputs of the above sensor/s.

3. The system according to claim 1 which includes a hardware processor comprising an AI module, and wherein a system of systems is used to increase reliability and accuracy of measurement, the system of systems including plural systems monitoring subjects from different distances and/or angles, and wherein the AI module is configured to use synchronized data from the plural systems to yield monitoring data regarding monitored subjects.

4. The system according to claim 1 wherein radar range and azimuth resolution are used to separate or differentiate, by processing, physically separated monitored subjects, thereby to yield measurement in a controlled environment which prevents reliability' degradation of monitored parameters.

5. The system according to claim 1 wherein a detection range of 10 or 20 or 30 or 40 or 50 or therebetween or more meters is provided, together with a radar range cell of 15 or 25 or 35 or 45 or 55 or 66 cm, or therebetween, thereby to enable vital signs measurement of plural monitored subjects, simultaneously, with minimal interference between them.

6. The system according to claim 1 wherein said radar sensor has a multi-beam mode of operation which allows simultaneous measurement of an entire volume having an update rate, thereby to lower influence of sporadic events such as noise, interference, and sporadic movement of monitored subject/s.

7. The system according to claim 1 wherein the system's field of view is mapped, using a grid mapping operation which generates position/s and angle/s of monitored subject/s.

8. The system according to claim 7 wherein said grid mapping operation is three dimensional in range, azimuth, and Doppler or phase change.

9. The system according to claim 1 wherein the radar sensor defines a radar range cell and wherein the radar range cell is modified between plural possible ranges in a quasi-arbitrary manner, thereby to reduce external or internal interferences of received-power.

10. The system according to claim 9 wherein said radar range cell is modified by changing an FMCW transmitted bandwidth, from one frequency to another within a range of possible frequencies.

11. The system according to claim 1 wherein outputs (aka radar readings) of said radar sensor are used to measure respiration, and wherein plural algorithms are used by a hardware processor for this purpose, and an AI module assigns weights to each algorithm to reflect that algorithm's abilities in different scenarios, using a database which the system may accumulate over time.

12. The system according to claim 1 wherein the system includes a thermal camera and wherein heart rate measurements are generated by the system including pointing the thermal camera at at least one Carotid artery of at least one monitored subject, and, accordingly, monitoring monitored subjects' Carotid artery blood flow, since blood flow through Carotid arteries changes the surface temperature of the neck, these heat changes being picked up by the thermal camera, blood through the Carotid arteries pulsates at heart rate, and wherein the heartbeat rate is then estimated by computing a rate of periodic heat change.

13. The system according to claim 1 wherein a wide-angle and/or visible light camera is used to determine movement type, if any, of monitored subject's and wherein, accordingly, measurement process quality is improved by identifying and computationally removing measurement anomalies associated with interferences and/or movement.

14. The system according to claim 1 wherein Artificial Intelligence software is used to assess measurements from plural system sensors (radar and/or thermal camera and/or wide angle camera) and to generate a time coherent presentation of relevant parameters.

15. The system according to claim 14 wherein said Artificial Intelligence software includes machine learning functionality which applies, to measured data, rules, learned on training data, for best evaluating vital signs from the measured data.

16. The system according to claim 15 wherein the system is used to detect which monitored subjects have a diagnosis and which do not, and wherein the training data comprises data from subjects known to have the diagnosis, and other subjects known not to have the diagnosis.

17. The system according to claim 15 wherein, as the system collects data, the system alerts of changes within the data, and the Artificial Intelligence software automatically changes processing modules accordingly.

18. The system according to claim 1 wherein the radar sensor has a staring mode thereby to cover a field of view simultaneously, yielding continuous radar coverage, without time gaps caused by steering the radar beam, thereby to support high update rates resulting in low false alarm rate and/or high detection probability, relative to lower update rates, higher false alarm rates and/or lower detection probabilities of scanning radars, and/or thereby to provide low MDV (minimal detectable velocity) or slow phase change and/or high azimuth accuracy, relative to higher MDV, and/or lower azimuth accuracies of scanning radars.

19. The system according to claim 18 wherein, using said staring mode, the system is configured to characterize interferences from different directions and disqualify or discount on that basis.

20. The system according to claim 18 wherein, using said staring mode, the system is configured for 3D target tracking using high range, azimuth and Doppler or phase change separation, relative to scanning radars.

21. The system according to claim 1 wherein the system includes a thermal camera and wherein air flow rate is sensed by the thermal camera and wherein lung capacity is computed using air flow rate and estimates, known to the system, of inhalation and/or exhalation cross sections of at least one of nose and mouth.

22. A system according to claim I which includes a thermal camera sensitive to at least one wavelength, and wherein body temperature measurements are generated by:
pointing the thermal camera toward at least one subject's body part, the body part typically including at least one of the subject's face, forehead, neck, palm of hand, thereby to generate a first reading;
pointing the thermal camera to a calibrated temperature generator, such as a black body element at said at least one wavelength, thereby to generate a second reading; and
generating a body temperature estimate by comparing said first and second readings.

23. A system according to claim 1 which includes a thermal camera which generates first heart rate measurements which are combined with second heart rate measurements provided by said radar sensor, thereby to provide an extremely accurate evaluation of heart rate and/or to remove at least one heart rate measurement artifact and/or at least one false heart rate measurement.

24. The system according to claim 21 wherein respiration rate is measured by the thermal camera using the inhaled and exhaled air flows.

25. The system according to claim 21 wherein the thermal camera generates first respiration rate measurements which are combined with second respiration rate measurements provided by said radar sensor, thereby to provide an extremely accurate evaluation of respiration rate and/or to disqualify at least one false measurement caused by subject motion and/or disqualify at least one false measurement caused by interference within the system's field of view.

26. A method for measuring medical data characterizing a subject to be monitored, the system including at least one radar sensor and at least one electro-optical sensor, and wherein the medical data, including pulse and/or respiratory rate and, optionally temperature of the subject to be monitored, are measured remotely, thereby providing standoff detection and reducing the risk of infection to medical personnel.

27. A computer program product, comprising a non-transitory tangible computer readable medium having computer readable program code embodied therein, said computer readable program code adapted to be executed to implement a method for measuring medical data characterizing a subject to be monitored, the system including at least one radar sensor and at least one electro-optical sensor, and wherein the medical data, including pulse and/or respiratory rate and, optionally temperature of the subject to be monitored, are measured remotely thereby providing standoff detection and reducing the risk of infection to medical personnel.
